(19) United States
(12) Patent Application Publication
(10) Pub. No.: US 2005/0084960 A1
(43) Pub. Date: Apr. 21, 2005

(54) Differentiation of human embryonic stem cells in avian embryos

(76) Inventors: Ron Goldstein, Jerusalem (IL); Nissim Benvenisty, Jerusalem (IL); Micha Drukker, Kfar Sirkin (IL)

Correspondence Address: BROMBERG & SUNSTEIN LLP, 125 SUMMER STREET, BOSTON, MA, 02110-1618, US

(21) Appl. No.: 10/497,728

(22) PCT Filed: Dec. 5, 2002
(86) PCT No.: PCT/IL02/00978

Related U.S. Application Data
(60) Provisional application No. 60/340,988, filed on Dec. 7, 2001.

(51) Int. Cl. C12N 5/08

(52) U.S. Cl. 435/366

(57) ABSTRACT

The invention relates to a method of preparing from human embryonic stem cells, differentiated cells suitable for transplantation, by introducing human embryonic stems cells into an avian host embryo. Also provided is a method of directing the differentiation of human embryonic stem cells by introducing them into a selected location in an avian host embryo, which dictates their differentiation pattern. The invention provides normal, transplantable differentiated human cells, e.g. progenitor and other cells, particularly neural cells. The invention also relates to therapeutic and diagnostic methods employing the differentiated cells of the invention.

### FIELD OF THE INVENTION

The present invention relates to a process of obtaining differentiated human embryonic stem cells using chick embryo, and to the differentiated cells obtained thereby and uses thereof.

### BACKGROUND OF THE INVENTION

Throughout this application any publications are referred to in parentheses. A full list of these publications appears at the end of the description, immediately preceding the claims. The contents of all of these references are fully incorporated herein by reference.

It is known in the prior art that human embryonic stem cells (ES) cells are pluripotent cells that can differentiate into a large array of cell types. When injected into immune-deficient mice, embryonic stem cells form differentiated tumors (teratomas) (Thomson et al., 1998; Amit et al., 2000; Reubinoff et al., 2000). However, embryonic stem cells that are induced in vitro to form embryoid bodies (EBs) provide a source of embryonic stem cell lines that are amenable to differentiation in the presence of growth factors into multiple cell types characteristic of several tissues. (Itskovitz-Eldor et al., 2000; Schuldiner et al., 2000). For example, ES cells become differentiated into neurons in the presence of nerve growth factor and retinoic acid (Schuldiner et al. 2001). Human ES cells and their differentiated progeny are important sources of normal human cells for therapeutic transplantation and for drug testing and development. Required by both of these goals is provision of sufficient cells that are differentiated to the extent, and are of the type, best suited for a patient's needs or the appropriate pharmacological test. Associated with this is a need for an efficient and reliable production of differentiated cells from embryonic stem cells.

As described above, directed differentiation of ES cells in vitro has been obtained for several cell types, especially neurons. However, there are several limitations to the in vitro approaches now used. First, some cell types have not yet been produced from human ES cells because they do not grow well in conventional monolayer cell culture. For example kidney, lung and other complex tissues require a 3-D structure that requires interaction with blood vessels. In addition, the human brain is composed of many different specific types of neurons. Studies to date that have generated neurons from human ES cells have not produced specific types of CNS neurons such as motorneurons, cortical pyramidal cells, mesencephalic dopamine neurons or PNS neurons such as dorsal root ganglion or sympathetic ganglion neurons. Therefore, it would is desirable to develop alternatives to conventional tissue culture for directing differentiation of human ES cells into normal human cells that are useful for transplantation and for pharmacological testing.

The developing vertebrate embryo has all of the appropriate growth factors and microenvironmental components for directing differentiation of all of its cells. This fact has been utilised to study the differentiation of adult human bone marrow stromal cells, by injecting them into sheep embryos and examining their developmental potential (Liechy et al., 2001).

The chick embryo is a well characterised and accessible system (it is much easier to obtain and operate on avian eggs than on mammalian embryos) for the study of inductive interactions and differentiation in development. The precise time and position of the development of all organ systems in the avian embryo has been determined over the past century, and the molecular details of many of the tissue interactions and growth factor and other inductive influences in differentiation have been worked out.

Several studies have shown that mammalian cells and tissues transplanted to avian embryos can respond to local cues and develop into tissues appropriate to their location in the host including the central nervous system (i.e. Fontaine-Perus et al. 1997) and peripheral neurons system (White and Anderson, 1999). This compatibility between mammalian and avian tissues and the accessibility to each and every developing tissue of the avian embryo provides an ideal system for producing differentiated human cells from human ES cells. Placing human ES cells into the appropriate microenvironment of the chick at the precise time and position that the endogenous chick tissues are differentiating, provides the correct combinations of growth factors and extracellular matrix components that could take years of effort to discover by trial and error in conventional tissue culture experiments.

After causing differentiation, the human cells could be recovered by either 1) killing off the chick cells using anti chicken MHC antibodies and complement, or 2) using genetically modified human ES cells that allow selection by antibiotics or using fluorescence activated cell sorting.

It is therefore an object of the present invention to provide a method for obtaining normal differentiated human cells and to provide the cells obtained thereby. It is a further object of the invention to use the differentiated cells in transplantation. It is yet another object of the invention to use normal differentiated cells in pharmacological tests. These and other objects of the invention will become apparent as the description proceeds.

### SUMMARY OF THE INVENTION

In a first aspect, the invention relates to a method of preparing from human embryonic stem cells, differentiated cells suitable for transplantation. The method of the invention comprises: (a) providing human embryonic stem cells; (b) introducing these embryonic stem cells into an avian host embryo; (c) providing suitable conditions for permitting in vivo differentiation of the cells into differentiated cells; and (d) isolating the differentiated cells from said host embryo.

The invention further provides a method of directing differentiation of human embryonic stem cells into specific differentiated cells suitable for transplantation. According to this embodiment, the method comprises the steps of: (a) providing human embryonic stem cells; (b) introducing the embryonic stem cells into a selected location in an avian host embryo, wherein the selected location determines the differentiation of said cells; (c) providing suitable conditions for permitting in vivo differentiation of said cells into specific differentiated cells; and (d) isolating said differentiated cells from said host embryo.

According to one preferred embodiment, the differentiated cells prepared by the method of the invention may be progenitor cells selected from the group consisting of neural progenitor cells, mesodermal progenitor cells, ectodermal progenitor cells and endodermal progenitor cells.

According to another preferred embodiment, the human embryonic stem cells prepared by the methods of the invention may be obtained from any of an embryoid body derived from a fertilized human egg, a parthenogenetic human oocyte and a chimeric cell. More specifically, where an embryonic stem cell is obtained from a chimeric cell, such cell may contain a somatic cell nucleus and cytoplasm from any one of an oocyte, a fertilized egg and a pluripotent stem cell.

According to another specifically preferred embodiment, the embryonic stem cells of the invention may be genetically modified cells, containing an exogenous DNA. More particularly, these genetically modified cells may be transformed or transfected with at least one expression vector carrying said exogenous DNA.

Such expression vector, according to a specific embodiment, may comprise as the exogenous DNA a nucleic acid sequence encoding any one of a selectable marker, a surface protein, a suicide gene and growth factor or a sequence suitable for knocking out the HLA locus. Alternatively, or additionally, the exogenous DNA may be a nucleic acid sequence encoding a therapeutic protein.

The expression vector comprised within the cells obtained by the methods of the invention, may comprise an exogenous nucleic acid sequence encoding a selectable marker. Such marker may be, for example, green fluorescent protein, lac Z, firefly Rennila protein, luciferase, red cyan protein, or yellow cyan protein. Alternatively, such selectable marker may be an antibiotic resistance protein.

The avian host embryo used by the methods of the invention is preferably a chick embryo. Preferably, such embryo is 40-45 hours old at the time of introducing the human embryonic stem cells.

Suitable conditions for permitting in vivo differentiation by the method of the invention may include incubation of said avian host embryo, transplanted or microinjected with the human embryonic stem cells, for an effective period of time, preferably a 1 to 5 days.

A second aspect of the invention relates to transplantable human differentiated cells. According to this embodiment, such cell is differentiated in vivo from undifferentiated human embryonic stem cell, in an avian host embryo. The transplantable human differentiated cells are in particular normal cells.

In one preferred embodiment of this aspect, the differentiated cells of the invention may be obtained by a method comprising the steps of: (a) providing human embryonic stem cells; (b) introducing these embryonic stem cells into an avian host embryo; (c) providing suitable conditions for permitting in vivo differentiation of the cells into differentiated cells; and (d) isolating said differentiated cells from the host embryo.

In a specifically preferred embodiment, the differentiated cells of the invention are obtained by a method of the invention.

The method of the invention of directing the differentiation of human embryonic stem cells into specific cells may be particularly used for obtaining neural progenitor cells. For this purpose, the provided human embryonic stem cells are introduced adjacent to the neural tube and notochord or within the neural tube/brain primordium of said avian host embryo, preferably by microsurgical transplantation or by microinjection.

In one embodiment, the human embryonic stem cells used by the methods of the invention may be cells genetically modified prior to their introduction to the avian host embryo to express a suicide gene. Alternatively, these human embryonic stem cells may be cells genetically modified prior to their introduction to the avian host to knockout the HLA locus.

The differentiation of the human embryonic stem cells into the desired neural progenitor cells within the avian host embryo may be determined by an immunoassay, for detecting a neuronal cell lineage specific marker. Such specific marker may be, for example, a general neural marker such as neurofilament protein or β-3-tubulin, or a marker of more specific neural types including tyrosine hydroxylase, gaba, and glutamic acid decarboxylase.

Thus, the invention further provides transplantable neural progenitor cells, which may be differentiated in vivo from undifferentiated human embryonic stem cell, in an avian host embryo. The invention further provides for specifically differentiated neural cells, such as, for example peripheral nervous system cells and particularly dorsal root ganglion cells.

According to a specifically preferred embodiment, the neural progenitor cell of the invention is preferably obtained by a method of directing differentiation of human embryonic stem cells into neural progenitor cells defined by the invention.

In a further aspect, the invention relates to a method of treating a pathological condition in a subject in need of such treatment. The method of the invention comprises administering an effective amount of human differentiated cells or of composition comprising the same to the subject. These cells are differentiated in vivo from undifferentiated human embryonic stem cell in an avian host embryo. In a preferred embodiment, the cells used for treatment may be the differentiated cells of the invention.

Still further, the invention provides for the use of differentiated cells in the preparation of a pharmaceutical composition for the treatment of a pathological condition. The cells used may preferably be cells differentiated in vivo from undifferentiated human embryonic stem cells in an avian host embryo. Most preferably, the cell used may be the cell as defined by the invention.

In yet another aspect, the invention relates to a pharmaceutical composition for treating a pathological condition in a subject in need of such treatment. The composition of the invention comprises as an active ingredient, the human differentiated cells of the invention.

The invention further provides for a method of treating a tissue-related pathological condition, particularly a neural-related condition, in a subject in need of such treatment. This specific method comprises administering an effective amount of human specifically differentiated cells, particularly neural progenitor cells or of composition comprising the same to the subject. The cells administered to the treated subject may preferably be specific cells differentiated in vivo from undifferentiated human embryonic stem cell, in an avian host embryo, particularly human neural progenitor cells of the invention.

Still further, the invention relates to the use of differentiated specific human cells, e.g. neural progenitor cells in the preparation of a pharmaceutical composition for the treatment of a tissue-related, e.g. neuronal-related pathological conditions. The cells used may be preferably differentiated in vivo from undifferentiated human embryonic stem cell in an avian host embryo. Most preferably, suitable cells for such use may be the cells of the invention.

A specific embodiment of the invention relates to a pharmaceutical composition for treating a tissue-related, e.g. neuronal-related pathological condition in a subject. The composition of the invention comprises as an active ingredient an effective amount of specific human differentiated cells, e.g. neural progenitor cells, differentiated in vivo from undifferentiated human embryonic stem cells, in an avian host embryo. Such cells may be, for sample, the neural progenitor cells of the invention.

In a further aspect, the invention relates to a method of screening for a substance having a therapeutic potential, comprising the steps of: (a) obtaining human differentiated cells differentiated in vivo from undifferentiated human embryonic stem cell in an avian host embryo; (b) contacting said cells with a test substance; and (c) detecting an end point indication. This end point indication is indicative of the therapeutic potential of said test substance.

Still further, the invention relates to a method of assessing the toxicological effect of an active, said method comprising the steps of (a) providing normal differentiated human embryonic cells as defined by the invention; (b) contacting said cells with said active agent; determining the effect of said agent on said cells; whereby any damage to said cells or part thereof indicates that said agent is toxic to human cells.

### BRIEF DESCRIPTION OF THE FIGURES

The foregoing features of the invention will be more readily understood by reference to the following detailed description, taken with reference to the accompanying drawings, in which:

FIG. 1A-1D shows a schematic representation of the surgery performed. The two or three most-recently formed somites of 12-20 somite chick embryos were crushed (1A) or removed (1B) and a colony of human ES (Embryonic stem cell) maneuvered into the space generated. 1C and 1D are photomicrographs of live embryos that received grafts of GFP-expressing human ES cells colonies 24 hours earlier. In 1C, the embryo was photographed in situ. In 1D, the embryo was pinned out in a dish for photography. Arrow—ES cells colony. NT—neural tube Som—somites.

FIG. 2A-2G shows differentiation and integration of human ES (embryonic stem) cells transplanted into chick somites.

2A. A low-magnification view of the region of a graft of GFP-expressing human ES cells into a damaged somite 4 days earlier. GFP-expressing cells were immunostained and nuclei stained with Hoechst. Two large masses of GFP+cells are present, one (ES) next to the host dorsal root ganglion (DRG), and the second, more laterally. In between these masses, GFP+cells are interspersed among chick cells. NT=neural tube.

2B. The area enclosed by the lower box in A shown at higher magnification includes a GFP+pseudostratified columnar epithelium (Epith). Above the epithelia is a mesenchyme composed of cells with large nuclei, some of which express lower levels of GFP.

2C. The area enclosed by the upper box in A, is shown at higher magnification. Individual and clumps of GFP+cells mingle with the chick tissue. GFP+cells (arrows) have larger nuclei than the cells of the host (filled arrowheads). The inset shows only the Hoechst staining, which allows easy distinction of human and chick nuclei by size.

2D. Tubules of cuboidal epithelium (Tub) derived from the grafted cells.

2E. A micrograph of a section where many GFP+human ES cells were present in the DRG (outlined) of the host. Some of these cells had processes suggestive of neurons (inset). NT=neural tube.

2F-2G. Human ES-derived cells that migrated to the vertebral arches (Vert), where they became elongated (arrow) and mingled with the chick perichondrial cells. Bars in A=150 m, B-D,G=40 m, E,F=80 m.

FIG. 3A-3J shows neural differentiation of human ES cells in the chick observed in sections through chick embryos receiving grafts of human ES cells that replaced epithelial somites. In panels 3A-3D, sections were stained with Feulgen and counterstained with Fast Green.

3A. A micrograph of an embryo two days after grafting at E4, in which distinct tubular structures have differentiated near the neural tube from the ES cells. The epithelium of human cells (*) is much darker staining and easily recognized even at this low magnification.

3B. At higher magnification part of the graft is identifiable as an early neural rosette-like epithelium (*), other cells make tubules of cuboidal epithelium (Tub).

3C. A low power micrograph from an embryo five days after grafting, stained also with Alcian Blue to visualize cartilage. A neural-rosette like structure (Ros) is enclosed within the vertebra (Vert).

3D. Numerous mitotic figures are observed adjacent to the lumen, two of which are indicated by arrows in this higher magnification image. NT=neural tube, DRG=dorsal root ganglion.

3E. A section through a graft stained with neuron-specific tubulin (green) and Hoechst nuclear dye (blue) is shown.

3F. Neuronal somata (arrows) at the basal aspect of the epithelium, and processes running perpendicular to its diameter (arrowhead) are clearly defined when the section is viewed at higher magnification. The hollow arrow points to a chick spinal nerve.

3G. A section through an operation where the grafted human ES cells fused with the host neural tube stained with antibodies to vertebrate neurofilament 200 (green), mammalian-specific neurofilament 160 (green) and Hoechst (blue). The graft is lateral to the chick's white matter (WM, compare with E), and the ventricular germinal zone (GZ) of the neural tube is distorted on the side of the graft (compare with the contralateral side and E).

3H. A projection of a Z-series of images made with a confocal microscope in an adjacent section to (G). The integration of the human (red) and chick (green) tissues is striking, with human axons (bold arrows) coursing through the chick white matter, and chick axons penetrating between the grafted human cells (arrows).

3I. Human neurons forming a ganglion-like structure (arrow). This section was stained with antibodies to HNK-1 (green) which is specific for the avian nervous system, and neuron-specific tubulin (red) which recognizes both human and chick neurons. The host DRG and nerve (hollow arrow) are stained yellow, since they are positive for both HNK-1 and neuron-specific tubulin. The nuclei of human neurons are larger even than the large neurons of the DRG nearby.

3J. Chick axons (bottom hollow arrow) traversing a glancing section through a rosette (Ros), which contains neuron-specific tubulin single-labeled axons and neurons (arrows) are observed in this section stained the same as (I).

### DETAILED DESCRIPTION OF THE INVENTION

In order to study the behavior of human ES cells in vivo, the inventors have transplanted ES cells in ovo to permit differentiation in early organogenesis-stage embryos. As shown by the following Examples, the process that has been specifically developed to demonstrate the present invention is to transplant green fluorescent protein (GFP) and neomycin resistance (Neo)-expressing human ES cells into chick embryos at the earliest stages of organ and tissue differentiation and formation. With this process it is possible to produce populations of specific types of human cells without the need for bovine or porcine biomaterials that are a potential source of disease. The microenvironment in the early chicken embryo causes the transplanted cells to differentiate, at least partially, due to inductive influences. The inventors have found neural, fibroblastic and possibly kidney (mesonephros) differentiation. By changing the position of the graft to be adjacent to specific developing organs of the chick host, the technique can provide heart, skeletal muscle, pancreas or any other human tissue cells.

As shown in Example 1, colonies of human ES cells were grafted into or in place of epithelial-stage somites of chick embryos at 1.5 to 2 days of development. The grafted human ES cells survived in the chick host, and were identified by using a selectable marker that was recognizable by optical, fluorescent or laser microscopy or other cell separation techniques. Examples of such markers are provided above and in the Example. For example, green fluorescent protein (GFP) was used as a marker to detect the embryonic stem cells that were introduced to the host avian embryo. Histological analysis showed that human ES cells are easily distinguished from host cells by their larger, more intensely staining nuclei. Some grafted cells differentiated en masse into epithelia, while others migrated and mingled with host tissues, including the dorsal root ganglion. Colonies grafted directly adjacent to the host neural tube produced primarily structures with the morphology and molecular characteristics of neural rosettes. These structures contain differentiated neurons as shown by expression of tissue specific markers such as β-3-tubulin and neurofilament expression in axons and cell bodies. Axons derived from the grafted cells penetrate the host nervous system, and host axons enter the structures derived from the graft. Other tissue specific markers discussed herein may be used to identify differentiated cells.

Example 1 shows that human ES cells transplanted in ovo survive, divide, differentiate and integrate with host tissues, and that the host embryonic environment can modulate their differentiation. The chick embryo may therefore serve as an accessible and unique experimental system for the study of in vivo development of human ES cells.

Thus, in a first aspect, the invention relates to a method of preparing from human embryonic stem cells, differentiated cells suitable for transplantation The method of the invention comprises: (a) providing human embryonic stem cells; (b) introducing these embryonic stem cells into an avian host embryo; (c) providing suitable conditions for permitting in vivo differentiation of the cells into differentiated cells; and (d) isolating the differentiated cells from said host embryo.

The invention further provides a method of directing differentiation of human embryonic stem cells into specific differentiated cells suitable for transplantation. According to this embodiment, the method comprises the steps of: (a) providing human embryonic stem cells; (b) introducing the embryonic stem cells into a selected location in an avian host embryo, which particular location determines the differentiation of said cells; (c) providing suitable conditions for permitting in vivo differentiation of said cells into specific differentiated cells; and (d) isolating said differentiated cells from said host embryo.

“Differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells.

According to one preferred embodiment, the differentiated cell prepared by the method of the invention may be a progenitor cell selected from the group consisting of a neural progenitor cell, mesodermal progenitor cell, ectodermal progenitor cell and endodermal progenitor cell.

“Embryonic stem cell” refers to a pluripotent cell type derived from any of the following:
(a) from the inner cell mass of a blastocyst from which embryonic bodies are formed providing embryonic stem cell monolayers (see W002/10347).
(b) Parthenogenesis (e.g. Cibelli et al. 2001).
(c) dedifferentiation of a somatic cell by the introduction of an effective amount of cytoplasm from a donor cell, i.e. an undifferentiated or substantially undifferentiated cell, e.g. an oocyte or cell from an inner cell mass of a blostomere, by methods such as micro-injection or use of liposomal delivery system into a recipient differentiated somatic cell (WO 01/00650).

Information regarding human ES cell lines suitable for use in the methods of the present invention is readily available at the NIH registry of human ES cells (http://escr.nih.gov/).

Thus, according to another preferred embodiment, the human embryonic stem cells used by the methods of the invention may be obtained from any of an embryoid body derived from a fertilized human egg, a parthenogenetic human oocyte and a chimeric cell. More specifically, where the embryonic stem cells are obtained from chimeric cells, such cells may contain a somatic cell nucleus and cytoplasm from any one of oocyte, a fertilized egg and a pluripotent stem cell.

According to another specifically preferred embodiment, the embryonic stem cells used by the methods of the invention may be genetically modified cells containing an exogenous DNA More particularly, these genetically modified cells may be transformed or transfected with at least one expression vector carrying said exogenous DNA.

As used herein, the term “transfection” means the introduction of a nucleic acid, e.g., an expression vector, into a recipient cell by nucleic acid-mediated gene transfer. Transfection may occur in vivo as well as in vitro. One result of transfection is to produce a genetically engineered cell.

As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides.

“Expression Vectors”, as used herein, encompass vectors such as plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

Plasmids are the most commonly used form of vector but other forms of vectors which serves an equivalent function and which are, or become, known in the art are suitable for use herein. See, e.g., Pouwels et al. (1988).

The vector is introduced into a host cell by methods known to those of skilled in the art. Introduction of the vector into the host cell can be accomplished by any method that introduces the construct into the cell, including, for example, calcium phosphate precipitation, microinjection, electroporation or transformation. See, e.g., Current Protocols in Molecular Biology, Ausuble, F. M., ed., John Wiley & Sons, N.Y. (1989).

The embryonic stem cells may be transfected with exogenous DNA under cell specific promoters (including embryonic stem cell specific promoters and differentiated cell specific promoters) or under promoters for house keeping genes expressed in all transfected cells regardless of differentiation prior to introduction into the chick embryo using the techniques described in Eiges et al., 2001.

Examples of promoters that are activated in embryonic stem cells are rex-1, oct-4, oct-6, SSEA-3, SSEA-4, TRA1-60, TR1-81, GCIM-2, alkaline phosphatase and Hes1 promoters. Examples of promoters that are active in differentiated cells include those generally non-coding nucleotide sequences located upstream from protein encoding for example, neurofilament heavy chain; cardiac promoters determine expression of cardiac proteins and actins in cardiac muscle cell, hematopoietic promoters determine expression of globin proteins including beta globin, a liver promoter regulates expression of albumin in hepatocytes, and a pancreatic promoter regulates expression of insulin. Other examples of promoters are those that regulate expression of nestin, tyrosine hydroxylase, dopamine beta hydroxylase, CD34, PGX-1, albumin, ISL-1 and ngn-3 and tub-3. These examples are not intended to be limiting. These promoters may be placed before a marker gene using recombninant DNA techniques known in the art so that the expression of the marker gene is controlled by the promoter.

Such expression vector according to a specific embodiment may comprise an exogenous nucleic acid sequence encoding any one of a selectable marker, a surface protein, a suicide gene and growth factor or a sequence suitable for knocking out HLA locus.

“Suicide sequence” or “suicide gene” in a cell is any DNA which, when activated as a result of an externally administered agent acting either directly on the DNA (or RNA) or on protein expressed by the DNA, results in apoptosis or damage to the cells containing the suicide sequence. Suicide genes can be under the control of a constitutive promoter or a tissue-specific promoter, for example a human ES cell specific promoter. When the cells are transplanted into a subject in vivo, the externally administered agent may be provided orally or parenterally, including by subcutaneous, intramuscular or intravenous injection or by transdermal means. Examples of suicide genes are inducible apoptotic genes and those encoding thymidine kinase, bacterial cytosine deaminase, inducible Diphtheria toxin, dexamethasone and the Tetracycline inducible system (Teton or Tetoff).

“Selectable markers” are DNA, RNA or protein that can be readily detected in cells and provide means of distinguishing those cells containing the marker from those lack it. Markers can be used to track cellular events in circumstances involving a changing environment. Markers can be intrinsic in the cells of interest or may be foreign (exogenous) and introduced into the cells to express proteins. For example, where foreign (exogenous) DNA encodes a marker, these are sometimes called reporter genes. “Reporter genes” are those genes that “report” the presence of particular cells and may include cell-specific enhancers and promoters that determine whether tissue-specific expression of a gene occurs, and how it is modulated. Reporter genes may be introduced into cells by transfection. Transfection of cells with genes encoding reporter proteins provides a means for tracking cells. Examples of reporter genes include green fluorescent protein, Lac Z, firefly Rennila protein, red, yellow or blue cyan fluorescent proteins or other fluorescent protein, including those found in marine animals. Other markers include antibiotic resistance proteins to protect cells against for example, neomycin, hygromycin, zeocine and puromycin.

In this way, marker proteins useful for distinguishing human cells from chick cells may be introduced into the human embryonic stem cells prior to their transplantation into chick embryos, and expressed either in the pluripotent cells and differentiated cells or more specifically in either pluripotent cells or particular differentiated tissue product originating from the human embryonic stem cells.

Thus, in another particular embodiment, the expression vector comprised within the cells used by the methods of the invention, comprises an exogenous nucleic acid sequence encoding a selectable marker. Such marker may be for example, green fluorescent protein, lac Z, firefly Rennila protein, luciferase, red cyan protein or yellow cyan protein. Alternatively, such selectable marker may be an antibiotic resistance protein.

Human ES stem cells may be manipulated in a manner suitable for differentiation and ultimately for transplantation into a human subject so as to remove cell surface antigens that induce tissue rejection. The genes of the major histocompatibility complex in embryonic stem cells were targeted by the present inventors so that the differentiated progeny are immunologically neutral. This was achieved by knock-out or inhibition (by anti-sense or dominant negative form overexpression or ribozymes) of beta-2-microglobulin or HLA-1 or HLA-11 or INF receptors. Any known method for inserting, deleting or modifying a desired mammalian gene with the transfection techniques described in Examples 1-5 can be employed. Methods and vectors for effecting gene knockout are the subject of numerous patents including U.S. Pat. Nos. 6,074,853, 5,998,144, 5,948,653, 5,945,339, 5,925,544, 5,869,718, 5,830,698, 5,780,296, 5,614,396, 5,612,205, 5,468,629, 5,093,257 all of which are herein incorporated by reference in their entirety.

In certain circumstances it is desirable that cells for transplantation contain “suicide” genes. Examples of “suicide” genes include a tetracycline inducible form of the diphtheria toxin (Maxwell, (1986)) and the bacterial cytosine deaminase (Pandha, (1999)). Any “suicide” gene known in the art may he used for the negative selection of transplanted cells in vivo or in vitro in the manner described herein. “Suicide” genes controlled by specific promoters will allow elimination of any of those cells in which the promoters are active.

The inventors have shown that human ES cell lines can be genetically engineered to constitutively express a suicide gene without changing the capacity of the cells to differentiate into a wide variety of tissues. For example, human embryonic stem cells can be transfected with an HSV-TK gene, which when expressed confers sensitivity to the FDA-approved drug Ganciclovir, allowing specific ablation of HSV-TK⁺ cells at concentrations non-lethal to other cell types. As expression of this gene causes sensitivity to the FDA approved pro-drug Ganciclovir (i.v. ganciclovir), it allows specific targeting of injected cells, allowing non-intrusive removal of grafts in case of unwanted side effects.

Concentrations of ganciclovir (10⁻⁵-10⁻⁷) which do not affect normal cells, kill HSV-TK transfected cells. Cells expressing particular genes in suitable quantities may be used in cell therapy in a subject to correct defective gene expression associated with a condition in the subject as an alternative to gene therapy. Examples of therapeutically beneficial proteins expressed by genes in differentiated cells derived from human embryonic stem cells include; growth factors such as epidermal growth factor, basic fibroblast growth factor, glial derived neurotrophic growth factor, nerve growth factor, insulin-like growth factor (1 and 11), neurotrophin-3, neurotrophin-4/5, ciliary neurotrophic factor, AFT-1, lymphokines, cytokines, enzymes-for example, glucose storage enzymes such as glucocerebrosidase, tyrosine hydroxylase.

The use of human ES cells that express GFP and Neo, allows separation of the human cells from the chick host when the desired effect is achieved: GFP expressing cells can be separated by fluorescence activated cell sorting, and chicken cells can be killed selectively by treatment with neomycin. In addition, chick cells may be selectively killed with an antibody against avian cell surface molecules in the presence of complement.

The avian host embryo used by the methods of the invention may be selected from the group consisting of chicken, turkeys, geese, ducks, pheasants, quails, pigeons and ostriches embryos, preferably, a chick embryo. Most preferably, such embryo may be 40-45 hours old, at the time of introducing human embryonic stem cells.

In yet another embodiment, suitable conditions for permitting in vivo differentiation by the method of the invention may include incubation of said avian host embryo containing the human embryonic stem cells, for an effective period, preferably, an effective period of 1 to 5 days under conventionally accepted conditions, particularly, in a humid environment, at 37-38° C. under normal atmospheric conditions.

A second aspect of the invention relates to differentiated human cells suitable for transplantation. According to this embodiment, such cells are differentiated in vivo from undifferentiated human embryonic stem cells, in an avian host embryo.

In one preferred embodiment of said aspect, the differentiated cells of the invention may be obtained by a method comprising the steps of: (a) providing human embryonic stem cells; (b) introducing these embryonic stem cells into an avian host embryo; (c) providing suitable conditions for permitting in vivo differentiation of the cells into differentiated cells; and (d) isolating said differentiated cells from the host embryo.

In a specifically preferred embodiment, the differentiated cell may be obtained by a method of the invention.

A major advantage of the method of the invention is the ability to direct the differentiation of the human ES cells towards a specific tissue, by selecting the location of transplantation in the host embryo.

Differentiation in-ovo of mammalian stem cells and embryonic tissues has been demonstrated, however, the avian embryo has not yet been used for study or manipulation of human stem cells/embryonic tissues. Since many aspects of stem cells have been found to be species specific (i.e. expression of SSEA markers, LIF dependence, ES colony morphology and doubling time differs for human and mouse ES), it was not not to be readily expected that human cells would respond to the avian environment. In addition, transplantation of human ES to the kidney capsule or subcutaneously in adult mice generates teratomas. These growths do not contain specifically cells related to the position of the graft, rather they are composed of cells of many different types that are generated in a random, stochastic manner.

Thus the inventors have succeeded in generating neural cells by introducing the human ES cells adjacent to neural tube and notochord, or within the neural tube/brain primordium. The cells can be introduced by microsurgical transplantation or by microinjection with a micropipette. The human neural cells can be progenitor neural cells, and specific subtype neural cells, particularly peripheral nervous system cells like dorsal root ganglion cells.

The introduction of the ES cells into the host embryo is to be adjusted to the intended differentiation. Thus, for neural cells, introduction of the ES cells was preferably at less than 48 hours after conception of the embryo. In order to obtain pancreas, introduction of the ES cells is preferably at about 72 hour post-conception, for heart, about 24 hours. Each desired tissue develops at a specific time. The removal of somites is simplest on embryos between 38-48 hours of incubation, but can be performed at earlier and later stages as well.

The differentiation of the human embryonic stem cells into the desired neural progenitor cells within the avian host embryo may be determined by an immunoassay which enables the detection of neuronal cells lineage specific markers. Such specific marker may be, for example, any one of neurofilament protein and tubulin, tyrosine hydroxylase, GABA, and glutamic acid decarboxylase.

Thus, the invention provides a neural progenitor cells suitable for transplantation. Such neural progenitor cells may be differentiated in vivo from undifferentiated human embryonic stem cells, in an avian host embryo.

According to a specifically preferred embodiment, the neural progenitor cell of the invention may be obtained by the method of the invention, wherein the embryonic stem cells are introduced adjacent to the neural tube and notochord or within the neural tube/brain primordium of the avian host embryo by microsurgical transplantation or by microinjection, preferably at less than 48 hours after conception thereof.

Likewise, based on histomorphology, the inventors have shown perichondial and possibly kidney (mesonephros) differentiation. Thus, the methods of the invention can also provide for the generation of human fibroblasts and mesonephros cells.

The method of the invention enables obtaining human differentiated cell which cannot at present be obtained in vitro, particularly cells of three dimensional and complex tissues. Examples of such tissues are, but not limited to, lungs, kidney, and gut.

Practical applications for the differentiated cells of the invention, include the following:
(a) Purified or semi-purified populations of differentiated human cells produced by the method of the invention can be used for replacement therapy in conditions as defined, including, to name but few, degenerative disease (e.g. Parkinson's disease, diabetes), stroke and traumatic injury. The potential of generating ES cells by therapeutic cloning from the patient, offers the possibility of the elimination of graft-rejection problems. The generated differentiated human cells can be transplanted in vivo, or used for example for tissue repair ex vivo, and then returned to the patient.
(b) Purified or semi-purified populations of normal differentiated human cells could be used for testing the efficacy of drugs on normal, non-transformed human tissues.
(c) Purified or semi-purified populations of normal differentiated human cells could be used for testing the damaging effect of various active agents on normal, non-transformed human tissues. Such tests can be applied to, for example, cosmetics, paints and varnishes and the like, food additives, OTC pharmaceutical preparations, agrochemicals and other preparations, and replace animal studies, experiments in human volunteers or tests performed on transformed cell lines, the results of which do not always coincide with experiments with normal cells.

Tissue and damage repair can be performed according to available techniques, such as those described in rodents. For example, differentiated mouse ES cells have been used successfully to treat a rodent model of Parkinson's disease (Kim et al. 2002).

Thus, in a further aspect, the invention relates to a method of treating a pathological condition in a subject in need of such treatment. The method of the invention comprises administering an effective amount of human differentiated cells of the invention or of a composition comprising the same to a specific location in the subject.

The term “subject” is defined here and in the claims as any living organism, more particularly a mammal, more particularly a human.

Still further, the invention provides for the use of the differentiated cells in the preparation of a pharmaceutical composition for the treatment of a pathological condition. The cells used are cells differentiated in vivo from undifferentiated human embryonic stem cells in an avian host embryo, in accordance with the invention.

The invention also relates to a pharmaceutical composition for treating a pathological condition in a human subject in need of such treatment, comprising as an active ingredient the human differentiated cells of the invention.

In particular, the invention provides for a method of treating a neuronal-related pathological condition in a human subject in need of such treatment. In this specific method, the cells administered are normal human neural progenitor cells, as provided by the invention. The cells may be more specific neural cells, such as dorsal root ganglion cells.

“pathological condition” describes a state that is manifested as different from normal and for which a human subject may seek treatment. Examples of conditions include cancers, such as late stage cancers including ovarian cancer and leukemia, diseases that compromise the immune system such as AIDS, and autoimmune diseases such as multiple sclerosis, diabetes mellitus, inflammatory bowel diseases such as Crohn's disease, systemic lupus erythematosus, psoriasis, rheumatoid arthritis, autoimmune thyroid disease and scleroderma, “neuronal related pathological conditions” which are conditions affecting the nervous system such as muscular dystrophy, Alzheimer's disease, Parkinson's disease, spinal cord injuries, liver diseases such as hypercholesterolemia, and other conditions for which replacement of damaged tissue is desirable such as in heart disease, cartilage replacement, burns, foot ulcers, gastrointestinal diseases, vascular diseases, kidney diseases, urinary tract disease and aging related diseases and conditions. The condition may be associated with defective genes, e.g. defective immune system genes, cystic fibrosis genes, or other genetic diseases.

In a further aspect, the invention relates to a method for screening for a substance having therapeutic potential comprising the steps of:
(a) obtaining human differentiated cells differentiated in vivo from undifferentiated human embryonic stem cells in an avian host embryo;
(b) contacting said cells with a test substance; and (c) detecting an end point indication. This end point indication is indicative of the therapeutic potential of said test substance.

As described above, the cells of the invention may be used for toxicological tests. Thus, in yet a further aspect, the invention relates to a method of assessing the toxicological effect of an active, said method comprising the steps of (a) providing normal differentiated human embryonic cells as defined by the invention, (b) contacting said cells with said active agent; and (c) determining the effect of said agent on said cells; whereby any damage to said cells or part thereof indicates that said agent is toxic to human cells.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Cell Culture

Human ES cell clones transfected with PGK-EGFP (Eiges et al., 2001) were grown as previously described (Schuldiner et al., 2000; Eiges et al., 2001). Briefly, ES cells were initially cultured on a mitomycin-C treated mouse embryonic fibroblast (MEF) feeder layer (obtained from day 13.5 embryos) in 80% KnockOut™ DMEM medium (Gibco-BRL), supplemented with 20% KnockOut™ SR—a serum-free formulation (Gibco-BRL), 1 mM glutamine (Gibco-BRL), 0.1 mM-mercaptoethanol (Sigma), 1% non-essential amino acids stock (Gibco-BRL), penicillin (50 units/ml), streptomycin (50 g/ml) and 4 ng/ml of basic fibroblast growth factor (bFGF). Before grafting, human ES cells were cultured for 1-3 days on gelatin-coated plates without MEFs.

PGK-EGFP plasmid or a PNT plasmid were introduced into the human ES cells (Tybulewicz et al., 1991) that contains two PGK promoters driving either neomycin resistance gene or the herpes simplex thymidine kinase gene. Transfection was performed using ExGen 500 (Fermentas). FACS analysis and cell sorting—FACS analysis of PGK-EGFP and PNT expressing cells was performed on a FACS Calibur system (Becton-Dickinson, San Jose, Calif.), according to their green fluorescent emission.

Microsurgery

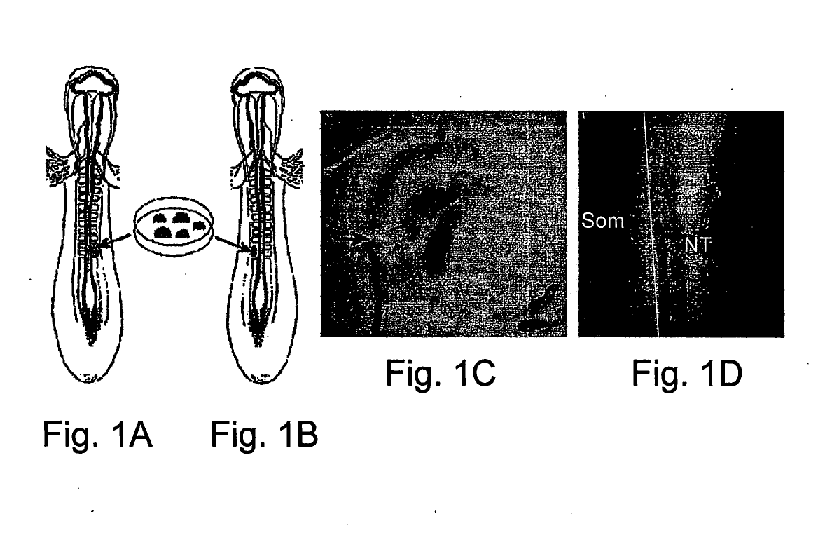

Fertile chicken eggs were incubated from 40-45 hours to obtain embryos of 10-20 somite pairs. A small amount of India ink was injected sub-blastodermally, and a tear made in the vitelline membrane above the 3-4 most recently formed somites. In some experiments, colonies were implanted laterally into somites manually damaged with a microscalpel (FIG. 1A). In other experiments, somites were removed after 5-10 minutes of enzymatic digestion, and the space formed was filled with a colony of human ES cells (FIG. 1B). Approximately 100-200 cells were implanted. Eggs were then sealed with Cellotape, and incubated an additional 1-5 days. Survival of the embryos was between 50-100% one day after surgery, at 5 days 20-50%. The grafted cells were found in sections in 50-75% of the surviving operations. In all, results presented are based on analysis of 15 grafts recovered from about 100 operations.

Histology and Immunocytochemistry

After the second incubation period, embryos were removed from the egg, rapidly fixed in buffered paraformaldehyde and embedded in paraffin. Serial sections were prepared and stained with antibodies using microwave antigen retrieval, or Feulgen and Fast Green. Antibodies used were rabbit anti-neurofilament (NF) 200 (N4142, Sigma), mammalian specific anti-NF 160 (clone 2H3,), rabbit anti-GFP, mouse anti-HNK-1 (ATCC) and anti-β-3-tubulin (TUJ1)+ or 5B8, Promega). Detection was performed with fluorescein/Texas Red or Alexa 488/594 conjugated secondary antibodies, and images captured digitally from an Olympus BX-60 compound or Biorad MRC 600 and 1024 confocal microscopes.

### Example 1

Human ES Cells Transplanted into Somitic Mesoderm Integrates into Chick Tissues

Colonies of human ES cells were micro-surgically grafted into the trunk region of 1.5 or 2 day-old (E1.5-E2) chick embryos (FIGS. 1A and 1B). One day after surgery the operation site was always visible. When GFP-expressing cells (Eiges et al. 2001) were implanted, they were clearly visible in the living embryo using fluorescence illumination (FIGS. 1C and 1D). The cells remained mostly as clumps, although individual cells could sometimes be observed migrating away from the site of implantation (not shown). The graft could be observed by fluorescence microscopy in some cases as long as four or five days post-surgery, after fixation and removal of overlying tissues (not shown).

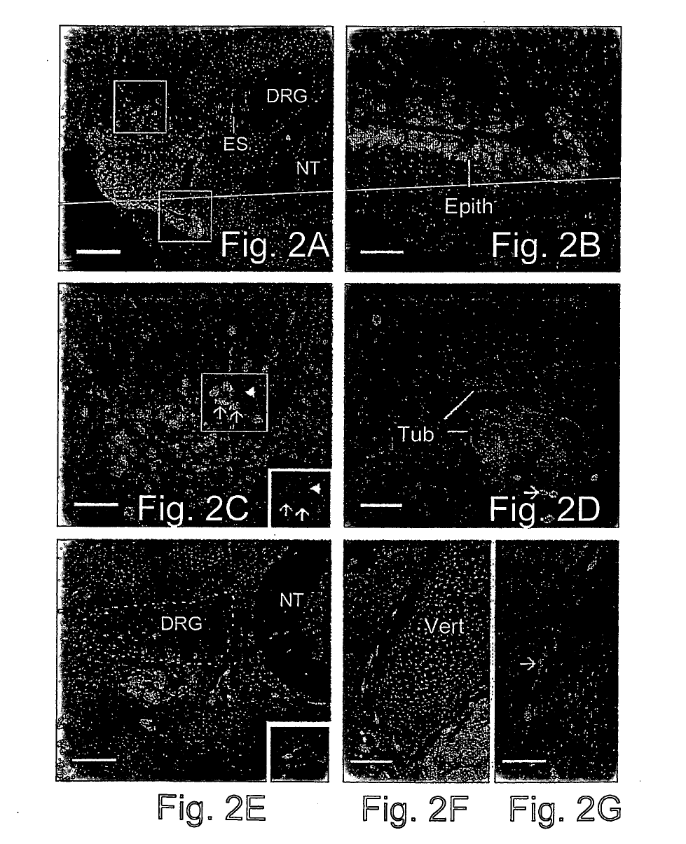

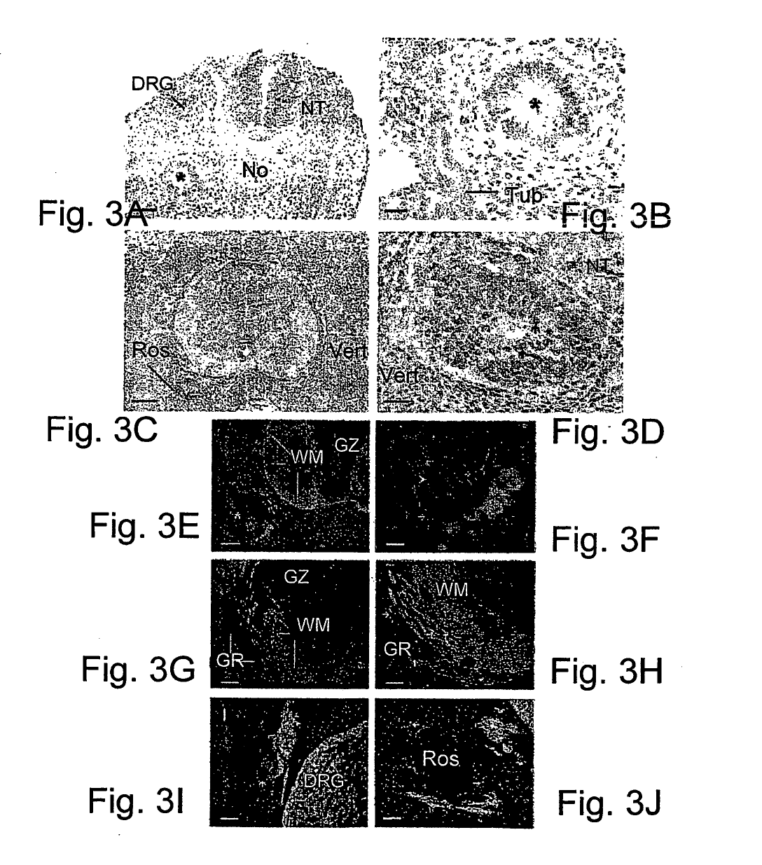

The somites give rise to multiple tissue types, including muscle, dermis and cartilage/bone. In addition, neural crest cells forming peripheral ganglia migrate through the somites after their epithelial/mesenchymal transformation. Therefore, the inventors initially implanted GFP-expressing human ES cells colonies into damaged somites to see if they would participate in the production of somitic or neural crest derivatives. Immunostaining for GFP demonstrated that some of the human ES cells remained as clumps (FIG. 2A), some cells formed distinct columnar (FIG. 2B) or cuboidal (FIG. 2D) epithelial structures while others mingled with the chick cells (FIGS. 2C, 2E-2G). Some human ES-derived cells incorporated into the host dorsal root ganglion (DRG) (FIG. 2E). Several of these cells had neuronal morphology, displaying axon-like processes (FIG. 2E, inset). In addition, elongated human ES-derived cells were observed lining the outside of the vertebral arch, apparently having contributed to the perichondrium (FIGS. 2F and 2G). In some (20%) preparations, structures resembling neural rosettes of teratomas developed from the grafted cells (see below). Although the human ES cells were implanted into the somite, morphological differentiation suggestive of the normal somitic derivatives (muscle, dermis and cartilage) was not observed. Anti-desmin and Alcian blue staining confirmed that these tissues had not formed from the ES up to 5 days after surgery (not shown). The lack of position-specific differentiation by human ES cells into these tissues could be due to the much more rapid organogenesis of avian compared to human embryos.

Hoechst staining revealed that all GFP+ cells contained larger nuclei that were usually more intensely stained than the surrounding chick cells (FIG. 2C, inset). Distinction of human from chick nuclei was also usually possible in preparations stained with Feulgen (FIGS. 3A-D) and hematoxylin and eosin (not shown). Similarly, grafted mouse cells can also often be distinguished from those of host chick embryos by nuclear staining (i.e. Fontaine-Perus et al., 1997). Grafted human ES cells, whether transplanted into or replacing somites, almost never developed a limiting capsule (1/20 operations). This is in striking contrast to teratomas formed from mouse and human ES cells that are bounded by a capsule that prevents ES-derived cells from integrating into host tissues.

### Example 2

Neuronal Differentiation of Human ES Cells Replacing Somitic Mesoderm

When colonies of human ES cells were implanted adjacent to the neural tube and notocord without intervening somitic mesoderm, epithelia reminiscent of neural rosettes were always (7 of 7 embryos analyzed) observed latero-ventral to the chick spinal cord (FIGS. 3A-3F). At embryonic days 6-7 these structures contained numerous mitotic figures that were localized primarily to their lumenal aspect (FIG. 3D). This arrangement of a stratified (or pseudo-stratified) epithelium with mitotic figures adlumenal and not basal, is characteristic of neural rosettes in human teratomas (Caccamo et al., 1989) and in the early vertebrate neural tube (see below). The neural rosette-like structures contained nuclei that were much larger than those of the host chick cells (FIGS. 3A-D).

Compared to human ES cells transplanted into damaged somites, this series of grafts contained many fewer individual cells that migrated away from the site of the surgery. There were also virtually no clumps of human ES cells with indeterminate morphology or columnar epithelia like those observed in the previous set of operations. However, cuboidal tubules (FIG. 3B) similar to those present in the damaged somite grafts (FIG. 2D) were sometimes present. The development of neural rosette-like structures in all preparations where colonies were transplanted adjacent to the host neural tube with no intervening tissues, suggests that the differentiation of human ES cells can be influenced by their local environment in the chick embryo.

Immunostaining for β-3-tubulin showed that human ES-derived rosette-like epithelia indeed contained neural cells with the pattern found in neural rosettes. Immunopositive neuronal somata were observed at the base, but not the lumen of the epithelia (FIGS. 3E and 3F). In addition, fine immunopositive processes were seen extending the diameter of the tubules (FIG. 3F). These processes were similar to those within which nuclei of neural-precursors in the embryonic neural tube migrate while in S-phase (Sauer, F. C. 1935).

In operations where human ES cells replaced somites, the inventors have also intentionally damaged the adjacent neural tube. This damage resulted in fusing of human ES-derived cells with the neural tube in 2 of 10 preparations (FIGS. 3G and 3H). Human axons were observed in the nascent white matter of the chick CNS, and chick axons coursed among the human ES cells (FIG. 3H). This was demonstrated by confocal examination of double immunostaining with an anti-neurofilament antibody that recognizes both mammalian and chick neurons, and a mammalian specific anti-neurofilament antibody. (Monoclonal antibody 2H3 was generated by Tom Jessel and obtained from the Developmental Studies Hybridoma Bank established under the auspices of the NICHD and maintained by the University of Iowa, Dept. of Biol. Sci., Iowa City, Iowa, 52242).

Immunostaining showed that some human neurons also developed from the human ES cells in structures that were not part of rosettes or the host CNS. Ganglion-like clumps of human neurons were observed near the DRG, as shown by double-staining specifically for chick nervous tissue with the HNK-1 antibody and generally for vertebrate neurons with β-3-tubulin antibodies (FIG. 3I). In addition, axons from the chick CNS were observed traversing the human ES-derived neural rosette-like structures (FIG. 3J).

All references cited herein are incorporated by reference.

### REFERENCES

Amit, M., Carpenter, M. K., Inokuma, M. S., Ciu, C.-P., Harris, C. P., Waknitz, M. A., Itskovitz-Eldor, J., and Thomson, J. A. (2000) Clonally derived human embryonic stem cell lines maintain pluripotency and proliferative potential for prolonged periods in culture. Dev. Biol. 227, 271-278.
Ausuble, F. M., ed.—Current Protocoles in Molecular Biology, John Wiley & Sons, N.Y. (1989).
Beuvais, J., Pla, P., Bernex, F., Dufour, S., Salamero, J., Fassler, R., Panthier, J.-J., Thiery, J. P., and Larue, L. (1999) A novel model to study the dorsolateral migration of melanoblasts. Mech. Dev. 89, 3-14.
Caccamo, D. V., Herman, M. M., Franfurter, A., Katestos, C. D., Collins, V. P., and Rubinstein, L. J. (1989) An immunohistochemical study of neuropeptides and neuronal cytoskeletal proteins in the neuroepithelial component of a spontaneous murine ovarian teratoma. American Journal of Pathology 135, 801-813.
Cibelli, J. B., Kiessling, A. A., Cuniff, K, Richards, C., Lanza, R. P. and West, M. D. (2001) Somatic Cell Nuclear Transfer in Humans: Pronuclear and Early Embryonic Development. The Journal of Regenerative Medicine 2, pg 25-31.
Eiges, R., Schuldiner, M., Drukker, M., Yanuka, O., Itskovitz-Eldor, J., and Benvenisty, N. (2001) Establishment of human embryonic stem cell-transfected clones carrying a marker for undifferentiated cells. Curr. Biol. 11, 514-518.

Fontaine-Perus, J. C., Halgand, P., Cheraud, Y., Rouaud, T., Velasco, M. E., Diaz, C. C., and Rieger, F. (1997) Mouse chick chimeras: a developmental model of murine neurogenic cells. Development 124, 3025-3036.

Itskovitz-Eldor, J., Schuldiner, M., Karsenti, D., Eden, A., Yanuka, O., Amit, M., Soreq, H., and Benvenisty, N. (2000) Differentiation of human embryonic stem cells into embryoid bodies comprising the three embryonic germ layers. Molecular Medicine 6, 88-95.
Kim, J. H., Auerbach, J. M., Rodriguez-Gomez, J. A., Velasco, I., Gavin, D., Lumelsky, N., Lee, S. H., Nguyen, J., Sanchez-Pernaute, R., Bankiewicz, K, McKay, R. (2002) Dopamine neurons derived from embryonic stem cells function in an animal model of Parkinson's disease. Nature. 418, 50-6.
Liechy, K. W., MacKenzie, T. C., Shaaban, A. F., Radu, A., Moseley, A. M., Deans, R., Marshak, D. R., and Flake, A. W. (2001) Human mesenchymal stem cells engraft and demonstrate site-specific differentiation after in utero transplantation in sheep. Nat. Med. 6, 1282-1286.
Maxwell (1986) Cancer Res., Vol. 46, pp. 4660-4664.
McDonald, J. W., Liu, X. Z., Qy, Y., Liu, S., Mickey, S. K., Turetsky, D., Gottlieb, D. I., and Choi, D. W. (1999) Transplanted embryonic stem cells survive, differentiate and promote recovery in injured rat spinal cord. Nat. Med. 5,1410-1412.
Pandha (1999) J. Clin. Oncol., Vol. 17, pp. 2180-2189.
Pouwels et al. Cloning Vectors: a Laboratory Manual (1985 and supplements), Elsevier, N.Y.
Reubinoff, B. E., Pera, M. F., Fong, C. Y., Trounson, A., and Bongso, A. (2000) Embryonic stem cell lines from human blastocysts:somatic differentiation in vitro. Nat. Biotech. 18, 399-404.
Rodriquez, et al. (eds.) Vectors: a Survey of Molecular Cloning Vectors and their Uses, Buttersworth, Boston, Mass (1988).
Sauer, F. C. (1935) Mitosis in the neural tube. J. Comp. Neurol. 62, 377-405.
Schuldiner, M., Yanuka, O., Itskovitz-Eldor, J., Melton, D. A., and Benvenisty, N. (2000) Effects of eight growth factors on the differentiation of cells derived from human embryonic stem cells. Proc. Natl. Acad. Sci. USA 97, 11307-11312.
Schuldiner, M., Eiges, R., Eden, A., Yanuka, O., Itskovitz-Eldor, J., Goldstein, R. S., and Benvenisty, N. (2001) Induced neuronal differentiation of human embryonic stem cells. Br. Res. 913, 201-205.
Thomson, J. A., Itskovitz-Eldor, J., Shapiro, S. S., Waknitz, M. A., Swiergiel, J. J., Marshall, J. J. and Jones, J. M. (1998) Embryonic stem cell lines derived from human blastocysts. Science 282, 1145-1147.
Tybulewicz et al., (1991) Cell, Vol. 65, pp. 1153-1163.
White P. M. and Anderson D. J. In vivo transplantation of mammalian neural crest cells into chick hosts reveals a new autonomic sublineage restriction. Development (1999) 126, 4351-63.

1-36. (canceled)

37. A method of preparing from human embryonic stem cells differentiated cells suitable for transplantation, which method comprises:
a) providing human embryonic stem cells;
b) introducing said embryonic stem cells into an avian host embryo;
c) providing suitable conditions for permitting in vivo differentiation of said stem cells into differentiated cells; and
d) isolating said differentiated cells from said host embryo.

38. A method of directing differentiation of human embryonic stem cells into specific differentiated cells suitable for transplantation, which method comprises the steps of:
a) providing human embryonic stem cells;
b) introducing said embryonic stem cells into a selected location in an avian host embryo which location determines the differentiation of said cells;
c) providing suitable conditions for permitting in vivo differentiation of said cells into specific differentiated cells; and
d) isolating said differentiated cells from said host embryo.

39. The method according to any one of claims 37 and 38, wherein said differentiated cell is a progenitor cell selected from the group consisting of neural progenitor cells, mesodermal progenitor cells, ectodermal progenitor cells and endodermal progenitor cells.

40. The method according to any one of claims 37 and 38, wherein said human embryonic stem cells are obtained from any one of an embryoid body derived from a fertilized human egg, a parthenogenetic human oocyte and a chimeric cell.

41. The method according to claim 40, wherein said chimeric cell contains a somatic cell nucleus and cytoplasm from any one of an oocyte, a fertilized egg and a pluripotent stem cell.

42. The method according to any one of claims 37 and 38, wherein said embryonic stem cells are genetically modified cells containing an exogenous DNA, transformed or transfected with at least one expression vector, and wherein said expression vector comprises a nucleic acid sequence encoding any one of a selectable marker, a surface protein, a suicide gene and growth factor or a sequence suitable for knocking out HLA locus; preferably said selectable marker is selected from the group consisting of green fluorescent protein, lac Z, firefly Rennila protein, luciferase, red cyan protein, yellow cyan protein and an antibiotic resistance protein.

43. The method according to any one of claims 37 and 38, wherein the avian host embryo is a chick embryo, which is 40-45 hours old at the time of introducing human embryonic stem cells, and wherein suitable conditions for permitting in-vivo differentiation are incubation of said avian host embryo comprising said human embryonic stem cells for an effective period of 1 to 5 days.

44. A transplantable differentiated cell, differentiated in vivo in an avian host embryo, from undifferentiated human embryonic stem cell.

45. A transplantable differentiated cell, wherein said cell is obtained by a method comprising the steps of:
a) providing human embryonic stem cells;
b) introducing said embryonic stem cells into an avian host embryo;
c) providing suitable conditions for permitting in vivo differentiation of said cells into differentiated cells; and
d) isolating said differentiated cells from said host embryo.

46. The differentiated cell according to claim 45, wherein said human embryonic stem cells are obtained from any one of an embryoid body derived from a fertilized human egg, a parthenogenetic human oocyte and a chimeric cell, and wherein said differentiated cell is a progenitor cell selected from the group consisting of a neural progenitor cells, mesodermal progenitor cells, ectodermal progenitor cells and endodermal progenitor cells; preferably said chimeric cell contains a somatic cell nucleus and cytoplasm from any one of an oocyte, a fertilized egg and a pluripotent stem cell.

47. The method according to claim 38, for directing differentiation of human embryonic stem cells into neural progenitor cells, which method comprises the steps of:
a) providing human embryonic stem cells;
b) introducing said embryonic stem cells adjacent to the neural tube and notocord or within the neural tube/brain primordium of said avian host embryo by microsurgical transplantation or by microinjection;
c) incubating said avian embryo comprising said human embryonic cells for at least 24 hours;
d) determining the differentiation of said neural progenitor cells; and
e) isolating said differentiated neural progenitor cells from said host embryo.

48. The method according to claim 47, wherein said human embryonic stem cells are cells genetically modified to express a suicide gene, prior to their introduction into said avian host.

49. The method according to claim 47, wherein said human embryonic stem cells are cells genetically modified to knockout the HLA locus, prior to their introduction into said avian host.

50. The method according to claim 47, wherein the differentiation of said cells is determined by an immunoassay for detecting neuronal cell lineage specific marker; preferably said marker is any one of general neural markers such as neurofilament protein and β-3-tubulin, and markers of more specific neural types including tyrosine hydroxylase, gaba, and glutamic acid decarboxylase.

51. A neural progenitor cell suitable for transplantation, which cell is differentiated in vivo from undifferentiated human embryonic stem cell, in an avian host embryo.

52. The neural progenitor cell according to claim 51, wherein said cell is obtained by a method comprising the steps of:
a) providing undifferentiated human embryonic stem cells;
b) introducing said embryonic stem cells adjacent to the neural tube and notocord or within the neural tube/brain primordium of said avian host embryo by microsurgical transplantation or by microinjection;
c) providing suitable conditions for permitting in vivo differentiation of said cells into neural progenitor cells;
d) incubating said avian embryo comprising said human embryonic cells for at least 24 hours;
e) determining the differentiation of said neural progenitor cells; and
f) isolating said differentiated neural progenitor cells from said host embryo.

53. The neural progenitor cell according to claim 51, wherein said cell is obtained by a method defined by any one of claims 47 to 50.

54. A method of treating a pathological condition in a subject in need of such treatment, comprising administering an effective amount of human differentiated cells or of composition comprising the same to said subject, which cells are differentiated in vivo from undifferentiated human embryonic stem cells in an avian host embryo.

55. The method according to claim 54, wherein said cells are as defined by any one of claims 44 to 46.

56. A pharmaceutical composition for treating a pathological condition in a subject comprising as an active ingredient human differentiated cells as defined by any one of claims 44 to 46.

57. A method of treating a neuronal-related pathological condition in a subject in need of such treatment, comprising administering an effective amount of human neural progenitor cell or of composition comprising the same to said subject, which cell is differentiated in vivo from undifferentiated human embryonic stem cell, in an avian host embryo.

58. The method according to claim 57, wherein said cells are as defined by any one of claims 52, and/or said cell is a neural progenitor cell obtained by the method defined by any one of claims 47 to 50.

59. A pharmaceutical composition for treating a neuronal related pathological condition in a subject comprising as an active ingredient a human neural progenitor cell differentiated in vivo from undifferentiated human embryonic stem cell, in an avian host embryo.

60. The composition according to claim 59, wherein said cell is defined by any one of claims 52, and/or said cell is a neural progenitor cell obtained by the method defined by any one of claims 47 to 50.

61. A method for screening for a substance having therapeutic potential comprising the steps of:
a) obtaining human differentiated cells differentiated in vivo from undifferentiated human embryonic stem cell in an avian host embryo;
b) contacting said cells with a test substance; and
c) detecting an end point indication, wherein said end point indication is indicative of the therapeutic potential of said test substance.

62. A method of assessing the toxicological effect of an active, said method comprising the steps of:
a) providing normal differentiated human embryonic cells as defined in any one of claims 44 to 46;
b) contacting said cells with said active agent;
c) determining the effect of said agent on said cells;
whereby any damage to said cells or part thereof indicates that said agent is toxic to human cells.
